(19) United States
(12) Patent Application Publication
(10) Pub. No.: US 2008/0255492 A1
(43) Pub. Date: Oct. 16, 2008

(54) Absorbent Fiber Material and Use Thereof in Wound Dressings

(76) Inventors: Jens Hoeg Truelsen, Helsingoer (DK); Brian Nielsen, Goerloese (DK); Niels Joergen Madsen, Alleroed (DK); Carsten Hoej, Vanloese (DK)

Correspondence Address: JACOBSON HOLMAN PLLC, 400 SEVENTH STREET N.W., SUITE 600, WASHINGTON, DC, 20004, US

(21) Appl. No.: 11/660,545

(22) PCT Filed: Aug. 19, 2005
(86) PCT No.: PCT/DK2005/000532

§ 371 Date: Mar. 13, 2008

(30) Foreign Application Priority Data
Aug. 19, 2004 (DK) PA 2004 01255

(51) Int. Cl. A61L 15/16; B32B 27/04; B32B 27/12

(52) U.S. Cl. 602/43; 442/118; 442/105

(57) ABSTRACT

An absorbent fibrous material comprising fibres prepared by melt spinning, solvent spinning, dry spinning or electro spinning of a polymer composition having a water absorption capacity of at least 10 g/g polymer composition and comprising a thermoplastic hydrophilic block copolymer as well as wound dressings comprising such absorbent fibrous material.

### BACKGROUND OF THE INVENTION

1. Field of the Invention

The present invention relates to absorbent fibrous material comprising fibres prepared by melt spinning, solvent spinning, dry spinning or electro spinning of a polymer composition having a water absorption capacity of at least 10 g/g polymer composition and comprising a thermoplastic hydrophilic block copolymer. In particular the present invention relates to non-woven fibrous material or a woven fibrous material comprising cartable fibres and/or spun fibres. The invention also relates to wound dressings comprising such absorbent fibrous material.

2. Background of the Invention

Fibrous materials are widely used as wound dressings or as absorbent elements in wound dressings on the advanced wound care market. These fibrous materials are water absorbent and gelling in order to be able to absorb and retain wound fluid without adhering to the skin. In addition, wound dressings of fibrous materials are conformable and flexible.

Commercially available wound dressings are based on Alginate and CMC (carboxy methyl cellulose) fibre materials.

However, there remains a need for flexible fibrous materials that are simple to produce and have good wound fluid absorption properties, improved gelling properties (no sticking to the wound bed), high wet strength, and low tendency to disintegrate during use.

GB 2 214 201 (corresponding to U.S. Pat. No. 4,923,454) describes an absorbent structure comprising a web of melt blown micro fibres, where the micro fibers are formed from a thermoplastic copolymer comprising a nylon component and a hydrophilic component, e.g. a block copolymer of nylon and polyethylene oxide diamine.

GB 1 488 776 describes fibres of a block copolymer comprising polyethylene terephthalate as the hydrophobic block and polyalkylene glycol as the hydrophilic block. The fibre is used in the core of polyester sheathed fibres and provides an increase in the water absorption properties of the synthetic fibres.

EP 341 870 describes a fibrous material comprising a mixture of fibres where one type of fibre is melt blown polyurethane fibres.

U.S. Pat. Nos. 4,820,577, 4,767,825 and 4,963,638 describe non-woven super absorbent webs comprising fibres of a super absorbent, thermoplastic polymeric composition, which are preferably prepared by melt blowing and co-forming. Other methods for the preparation of the fibres are mentioned.

WO 00/391179 citing the above US patents, describes super absorbent and elastic polymeric materials comprising soft segments and hard segments as well as the preparation of non-woven webs from this polymeric material by melt blowing, co-forming and spun bonding.

Melt-blowing of fibres as described above, results in a fibrous material which is not cartable and which may not be spun, because melt-blowing causes the fibres to form a non-woven fibrous material where the fibres are melted together.

Cartable fibres have the advantage that a broad range of different non-woven and woven structures may be manufactured. Optimised non-woven materials with respect to perception, absorption and slough handling may therefore be manufactured form cartable fibres.

WO 03/086234 describes electro-spinning of a thermoplastic, hydrophilic polyurethane polymer (Tecophilic). The fibrous materials obtained have a water absorption capacity of about 500%. Addition of a water absorbent material to the fibres improves the water absorption capacity considerably.

Hydrophilic block polymers as mentioned above typically consist of blocks with a large difference in their melting temperature/glass transition temperature, which makes the processing of the polymer difficult since the material needs to be processed at a temperature where the material with the lowest melting temperatures/glass transition temperature is form stable. Also, the hydrophilic material may absorb water if precautions are not taken. Absorbed water may evaporate during the processing and cause week points (gas bubbles) in the fibers. Finally, some hydrophilic segments may be hard and brittle causing problems during processing of the material.

It has now surprisingly been found that it is possible to spin flexible and elastic fibres, in particular such fibres that are cartable, and mono or multifilament fibres from thermoplastic hydrophilic block copolymers having a much higher water absorption capacity, than the polymer used in WO 03/086234.

### SUMMARY OF THE INVENTION

Accordingly, the present invention relates to an absorbent fibrous materials comprising fibres prepared by melt spinning, solvent spinning, dry spinning or electro spinning of a polymer composition having a water absorption capacity of at least 10 g/g polymer composition and comprising a thermoplastic hydrophilic block copolymer.

In a particular embodiment, the invention relates to an absorbent non-woven fibrous material comprising cartable fibres or a woven fibrous material comprising spun fibres wherein said fibres are made from a polymer composition having a water absorption capacity of at least 10 g/g polymer and comprising a thermoplastic hydrophilic block copolymer.

Suitable, the absorbent fibrous material swells and gels upon absorption of fluid, which makes it particularly useful in wound dressings.

Thus, the invention also relates to wound dressings consisting of an absorbent fibrous material as above, or comprising an absorbent fibrous material as above in addition to other elements.

### DETAILED DESCRIPTION OF THE INVENTION

Whenever a figure for the water absorption capacity of a fibrous material is given herein, it is the water absorption capacity measured by making a circular sample of the non-woven or woven fibrous material, weighing the sample (W1) and placing the sample in a petri dish with excess solution A (142.5 nM NaCl and 2.5 nM CaCl₂). The material is left 24 hours at 37° C. The sample is removed and allowed to drip for 30 sec and thereafter the sample is weighed (W2). Water absorption is calculated as W2−W1/W1.

Whenever a figure for the water absorption capacity for a polymer composition is given herein, it is the water absorption capacity measured by using following procedure: A suitable amount of material is heated to a temperature above the highest glass transition/melting temperature of the block copolymer and pressed to a sheet. An oblate with a well-defined diameter is punched out of the sheet and used for the absorption capacity measurements. The oblate was afterwards placed in a petri dish containing Solution A (see above). The weight increase of the oblate was followed by removing the oblate from petri dish, allowing exciding Solution A to drip off and weighing the oblate at predetermined time intervals. This procedure was continued until no further increase in weight was observed. Whenever used herein, cartable fibres mean fibres that may be carted and processed into a non-woven or woven fibrous material. Cartable fibres may suitably be obtained by melt spinning, solvent spinning or dry-spinning.

As used herein spun fibres means fibres that have been spun into yarns or treads, or monofilament fibres that have been wounded into multifilament fibres, yarns or treads. Spun fibres may suitably be obtained by melt spinning, solvent spinning or dry spinning of the polymer composition and then processed (cut, carted, etc) before they are spun into the yarns or treads, or wounded into the multifilament fibers, yarns or treads from which the woven material is made.

As used herein a hydrophilic block copolymer is a block copolymer, which absorbs water.

An amphiphilic block copolymer is a hydrophilic block copolymer as above containing both hydrophobic as well as hydrophilic polymer blocks.

The amphiphilic block polymers used to prepare the fibrous material according to the invention suitably consist of a non-polar polymeric chain(s) covalently coupled to a polar polymeric chain(s).

According to one embodiment of the invention the hydrophilic block copolymer is thermoplastic hydrophilic or amphiphilic polyurethane.

In one particularly preferred embodiment of the invention, the hydrophilic block copolymer is an hydrophilic or amphiphilic polyurethane block copolymer, such as polyethyleneoxide based polyurethanes.

Useful thermoplastic hydrophilic polyurethanes may be selected from Tecogel TG 2000 (water absorption capacity: 13 g/g), Estane T5410, Tecophilic, HydroMed D640™ and HydroSlip C™ (water absorption capacity 20 g/g).

According to a further embodiment of the invention the hydrophilic block copolymer is a thermoplastic amphiphilic block copolymer, wherein the hydrophobic block(s) comprises monomers selected from monovinyl aromatic monomers, ethylenically unsaturated monomers, and wherein the hydrophilic block(s) comprises monomers selected from ethylenically unsaturated monocarboxylic and dicarboxylic acids, such as acrylic acid, methacrylic acid, itaconic acid, maleic acid and fumaric acid; and monoalkyl esters of dicarboxylic acids of the type mentioned above with alkanols, preferably alkanols having from 1 to 4 carbon atoms and their N-substituted derivatives; amides of unsaturated carboxylic acids, such as acrylamide, methacrylamide, N-methylolacrylamide or methacrylamide, and N-alkylacrylamides; ethylenic monomers containing a sulphonic acid group and ammonium or alkali metal salts thereof, for example vinylsulphonic acid, vinylbenzenesulphonic acid, alpha-acrylamidomethyl-propanesulphonic acid and 2-sulphoethylene methacrylate; amides of vinylamine, especially vinylformamide or vinylacetamide; and unsaturated ethylenic monomers containing a secondary, tertiary or quaternary amino group, or a heterocyclic group containing nitrogen, such as, vinylpyridines and vinylimidazole, aminoalkyl-(meth)acrylates and aminoalkyl (meth)acrylamides such as dimethylaminoethyl acrylate or methacrylate, di-tert.butylaminoethyl acrylate or methacrylate and dimethylaminoacrylamide or dimethylaminomethacrylamide.

The hydrophobic block(s) of the amphiphilic block copolymer may suitably be polystyrene, a poly alpha-olefin such as polyethylene, polypropylene, poly-1-butene or polyisobutylene, a poly acrylate, a polyvinylether, a polyacetate, a polysiloxane, or a hydrophobic polyester.

The hydrophilic block(s) of the amphiphilic block copolymer may suitably be any type of polymer that will be able to absorb significant amounts of water. If taken alone, the hydrophilic block is water-soluble or at least highly water absorbing. Suitable hydrophilic block(s) in the amphiphilic block copolymer is PEG (polyethylene glycol), PVP (polyvinyl pyrrolidone), polyacrylic acid, salts of polyacrylic acid, salts of polymers of composed with acids such as maleic acid, polyvinyl alcohol, hydrophilic polyurethanes, or carbohydrates or gelatins.

According to a preferred embodiment, the hydrophobic block(s) comprises, or consist of monovinyl aromatic monomers, containing from about 8 to about 18 carbon atoms, such as styrene, alpha-methylstyrene, vinyltoluene, vinylpyridine, ethylstyrene, t-butylstyrene, isopropylstyrene, dimethylstyrene, and other alkylated styrenes and the hydrophilic block(s) comprises or consist of ethylenically unsaturated carboxylic acid monomers, such as acrylic acid, methacrylic acid, itaconic acid, maleic acid and fumaric acid.

Suitable, the hydrophobic block comprises styrene monomers and the hydrophilic block comprises monomers of acrylic acid or ethylene oxide, more preferred the hydrophobic block(s) is polystyrene and the hydrophilic block(s) is polyacrylic acid or polyethylene glycol.

According to another embodiment of the invention, the hydrophobic block(s) comprises or consist of ethylenically unsaturated monomers, such as butadiene, chloroprene, (meth)acrylic esters, vinyl esters, such as vinyl acetate, vinylversate and vinyl propionate; or vinyl halides, such as vinylchloride and vinyl nitriles and the hydrophilic block(s) comprises or consist of ethylenically unsaturated carboxylic acid monomers, such as acrylic acid, methacrylic acid, itaconic acid, maleic acid and fumaric acid.

Suitable, the hydrophobic block(s) comprises a (meth)acrylic ester monomer, such as methylmethacrylate, and the hydrophilic block(s) comprises methacrylic acid, and more preferred the hydrophobic block(s) is a poly(meth)acrylic ester, such as polymethylmethacrylate, and the hydrophilic block(s) is polymethacrylic acid.

The term (meth)acrylic esters means esters of acrylic acid and of methacrylic acid with optionally halogenated, e.g. chlorinated or fluorinated, C₁-C₁₂ straight or branched alcohols, preferably C₁-C₈, alcohols. Examples of such esters are methyl acrylate, ethyl acrylate, propyl acrylate, n-butyl acrylate, isobutyl acrylate, 2-ethylhexyl acrylate, tert.butyl acrylate, methyl methacrylate, ethyl methacrylate, n-butyl methacrylate and isobutyl methacrylate.

Suitable vinyl nitriles are those having from 3 to 12 carbon atoms, such as, in particular, acrylonitrile and methacrylonitrile.

Amphiphilic block copolymers for use in accordance with the present invention may be functionalised for further reactions like graft copolymerisation, cross-linking or for further polymerisation by inclusion of suitable functional groups. The functional groups may be attached to the ends of the main chains or as side chains. The functional groups may e.g. be unsaturated vinyl groups containing double bonds for further polymerisation. The functional groups may furthermore be photo initiators attached to the block copolymers for UV-polymerisation or for cross-linking. The functional groups may be hydroxyl, primary or secondary amine groups for further reactions with isocyanate for the formation of polyurethane based block copolymers or for cross-linking with isocyanate.

The hydrophobic block of the block-copolymer will constitute separate physically cross-linked domains being incompatible with the hydrophilic phase.

Amphiphilic block copolymers having long hydrophobic end blocks as di, tri, multi or star block copolymers may improve the cohesion within the fibre dramatically due to the physical cross-linking of the amphiphilic block copolymers and a high cohesion may be maintained during hydration and water absorption.

The hydrophilic block preferably has a minimum molecular weight of about 500 in order to be able to form separate hydrophilic domains. Preferably the molecular weight is higher than 1000 in case of end blocks and 5000 in case of midblocks. According to one embodiment of the invention, the hydrophilic block copolymer may suitably be a tri-block copolymer of the type A-B-A, wherein A is the hydrophobic block(s) and B is the hydrophilic block.

Alternatively, the block copolymer according to the invention has the form of a multi block or three or multi arm star-shaped block copolymer structure.

Where the hydrophilic or amphiphilic block copolymer is a polyurethane, the polymer may have more than two hydrophobic blocks and more than two hydrophilic blocks (ABABABABA).

Preferred amphiphilic block copolymers to be used in fibres in accordance with the present invention are such wherein the hydrophobic A domain is an end block of a mono vinyl aromatic homo polymer. Preferably the amphiphilic block copolymer is one having at least two different molecular weight blocks in the copolymer (e.g., a hydrophobic A block of about 1000 to about 50,000 number average molecular weight and a hydrophilic B block of about 1000 to about 500,000 number average molecular weight).

Suitable hydrophilic block copolymers according to the invention are amphiphilic polyurethanes, such as Estane T 5410, Tecogel 500, Tecogel 2000, Tecophilic HP-93A-100, HydroMed D640™ and HydroSlip C™, a poly(styrene-b-acrylic acid-b-styrene) block copolymer, a poly(methyl methacrylate-b-methacrylic acid-b-methyl methacrylate) block copolymer, or a poly(styrene-b-ethylene oxide-b-styrene) block copolymer.

Suitably the polymer composition according to the invention has a water absorption capacity between 10 and 30 g/g polymer composition, more preferred the polymer composition has a water absorption capacity between 10 and 25 g/g polymer composition, even more preferred the polymer composition has a water absorption capacity between 10 and 20 g/g polymer composition.

According to a further embodiment of the invention, the polymer composition according to the invention has a water absorption capacity between 15 and 30 g/g polymer composition, more preferred the polymer composition has a water absorption capacity between 15 and 25 g/g polymer composition. In a further embodiment, the polymer composition has a water absorption capacity between 15 and 20 g/g polymer composition.

In one embodiment, the polymer composition used according to the invention comprises or consists of a thermoplastic hydrophilic block copolymer having in itself a water absorption capacity with in the above mentioned ranges.

The cartable or spun fibres according to the invention may be made from a polymer composition comprising or consisting of one or more thermoplastic hydrophilic or amphiphilic block copolymers or a polymer composition comprising a blend of one or more hydrophilic or amphiphilic block copolymer(s) and other polymers (homopolymers, heteropolymers, copolymers), preferably hydrophilic homopolymers, heteropolymers or random copolymers.

In one example, a hydrophilic polymer, such as PVP, is blended with the amphiphilic block copolymer in order to increase hydrophilic properties. In another example a cohesion-promoting component such as polyacrylic acid or polycarboxylic acid or acrylic acid copolymers or associative thickeners may be added to the amphiphlic polymer before the fibres of the invention are made. In a third example, polyethyleneglycol is added to hydrophilic or amphiphilic block copolymer in order to adjust viscosity before making the fibres.

The polymer composition for making the fibres of the invention may also contain plasticisers. Suitable plasticizers for hydrophilic fibres are e.g. PEG 400, propylene glycol, dipropylene glycol, glycerol, glycerol diacetate (diacetin), glycerol triacetate (triacetin), triethyl citrate (Citrofol BII), Santicizer 141, Santicizer 148, Santicizer 261, Sebacic acid and tributyl citrate (Citrofol BI). In some instances addition of hydrophobic plasticizers may also be useful.

The fibrous material according to the invention may consist of fibre(s) comprising or consisting of a hydrophilic block copolymer as described above or it may contain a mixture of fibres, including fibres made from other polymer compositions.

Fibres made from other polymers compositions, which may form part of the absorbent fibrous material of the invention may be any fibre. Suitably, these fibres are gel-forming fibres, for examples fibres of polysaccharides, such as alginate, chitin, chitosan or CMC.

Other types of fibers which may be used together with the fibers comprising or consisting of a thermoplastic hydrophilic block copolymer as described above may be fibers giving strength or elasticity, such as nylon, cellulose, viscose, polyethylene, polypropylene or the like or super-absorbent fibres, such as polyacrylate fibres.

In one preferred embodiment of the invention, the absorbent fibrous material according to the invention contains fibres comprising the hydrophilic block copolymer as well as gel forming fibres.

Suitably, the absorbent fibrous material according to the invention comprises at least 20% w/w of fibres comprising or consisting of thermoplastic hydrophilic block copolymer, more preferred at least 30% w/w and most preferred at least 50% w/w fibres comprising or consisting of the thermoplastic hydrophilic block copolymer.

According to the invention, the absorbent fibrous material has a water absorption capacity of more than 10 g/g, suitably more than 12 g/g, more suitably more than 15 g/g, even more suitable more than 20 g/g, preferably more than 25 g/g or more than 30 g/g fibrous material. Preferred, the fibrous material comprises a hydrophilic or amphiphilic polyurethane as the hydrophilic block copolymer and has a water absorption capacity of between 15 and 35 g/g fibrous material.

The fibres in the absorbent fibrous material according to the invention may be prepared by melt spinning, solvent spinning, dry spinning or electro spinning.

Melt spinning is well known in the art and involves extrusion while the polymer composition is heated to a temperature above its melting temperature/glass transition temperature. The polymer is drawn, cooled and wound up using appropriate drawing and bubbling rates.

The principles of solvent and dry spinning are also well known to the skilled person.

In electro spinning a droplet formed by a polymer solution or polymer melt is applied to a strong electrical field. The die is acting as one of the electrodes and the counter electrode is a grounded metal sheet attached to a collector for the filament, which can be either stationary or rotating. When the electrical force at the surface of a polymer solution or polymer melt overcomes the surface tension and the viscous force a charged jet is ejected. The electrical forces elongate the jet many times and the jet becomes very thin. Ultimately the solvent evaporates, or the melt solidifies. The results are very long, thin nano-fibers, which are colleted on the counter electrode. US patent application nos. 20040061253, 20030201579 and 20020089094 as well as the references cited in these documents describes the principles of electro spinning.

The fibre diameter obtained depend on the processing parameters such as the surface tension, the viscosity and conductivity, the concentration of the solutions and flow rates, molecular weights and distribution, applied fields and electrode configurations.

Fibres with diameters in the range of a few nanometers to few micro-meters may be are obtained.

Drugs, proteins or functional polymers can be incorporated into the polymer composition during the spinning process by using mixtures and one die, or using concentrically twin dies.

The polymer composition may also be co-extruded by concentrically twin dies. This option may be particularly advantageous where it is desired to improved the strength of the fibre by having a strong fibre in the core of the fibre and the hydrophilic or amphiphilic block copolymer as a hydrophilic coating on the core fibre.

Suitable, the absorbent fibrous material according to the invention is a non-woven fibrous material. Preferably, the non-woven fibrous material of the invention is prepared from melt spun, solvent spun, dry spun or electro spun fibres, most preferred from melt spun fibres. Preferably, the fibres are cartable and may (or may not) have been subjected to a carting process during the process of forming the non-woven fibrous material.

The non-woven fibrous material according to the invention may thus be prepared by conventional methods such as crimping and carding the fibres of the invention. Finally the fibres may be cross-folded and needled.

As an example water absorbing and swelling polyurethane fibers may be produced by melt spinning of Tecogel 2000. The obtained fibers are treated (crimped) and cut to app. 5 cm length. These cut stable fibres are then carded, layered and cross folded and needled to a layer weighing 160 gsm. The Non-woven fiber material may be cut to 10×10 cm packed, and β-irradiated (sterilised).

Alternatively, the fibrous material according to the invention is a woven fibrous material mad from spun fibres. The spun fibres may be prepared by melt spinning, electro-spinning, dry spinning or solvent spinning. The fibres prepared by melt spinning, dry spinning or solvent spinning may be carted and/or processed into stable cut fibres or otherwise processed and thereafter spun and used to make the woven material. Monofilaments made by melt spinning, solvent spinning or dry spinning may also be used directly for making a woven fabric or several mono filaments may be wounded into a yarn or thread (multifilament) and used to make a woven fabric.

Monofilaments may also be cut into stable fibres, which are then processed into non-woven or spun into yarns or threads and further processed into a woven material.

The fibres of the invention may also contain and/or carry on their surface, substances such as pharmacologically or biologically active compounds, deodorising agents, pigments, wound healing associated indicator(s) such as indicators of pH, partial pressure of O₂, temperature, radical mechanisms or biotechnological assays, e.g. indicating formation of collagen.

This opens for a combined medical treatment of wounds and an easy and sterile application of the active ingredients. Incorporating active ingredients into the absorbent fibrous material enables local administration of active compounds in a wound. The active ingredient may suitable be a cytochine such as growth hormone or a polypeptide growth factor in which may exercise an effect on the wound, other medicaments such as bacteriostatic or bactericidal compounds, e.g. iodine, iodopovidone complexes, chloramine, chlorohexidine, silver salts such as Alphasan 2000 or 5000 (sodium hydrogen silver-zirconiumphosphate), sulphadiazine, silver nitrate, silver acetate, silver lactate, silver sulphate, silver-sodium-thiosulphate, silver chloride or silver complexes, zinc or salts thereof, metronidazol, sulpha drugs, and penicillins, tissue-healing enhancing agents, e.g. RGD tripeptides and the like, proteins, amino acids such as taurine, vitamins such ascorbic acid, enzymes for cleansing of wounds, e.g. pepsin, trypsin and the like, proteinase inhibitors or metalloproteinase inhibitors such as ethylene diamine tetraacetic acid, cytotoxic agents and proliferation inhibitors for use in for example surgical insertion of the product in cancer tissue and/or other therapeutic agents which optionally may be used for topical application, pain relieving agents such as lidocaine, chinchocaine or non-steroid anti-inflammatory drugs (NSAIDS) such as ibuprofen, ketoprofen, fenoprofen or declofenac, emollients, retinoids or an agents having a cooling effect.

The absorbent fibrous material of the invention is useful, for example, as an absorbent material in wound dressings. The absorbent fibrous material of the invention may be the sole element in the wound dressing or the wound dressing may contain other elements, such as a liquid impervious backing layer, an adhesive border etc.

The absorbent fibrous material according to the invention may be used, as the absorbent element(s) in any know wound dressing.

Highly or super absorbent dressings comprising fibrous materials according to the invention may also be used to absorb other body fluids, e.g. in connection with leaks from stomas, use of catheters etc.

In the following the invention is illustrated by way of examples. However, these examples may not be construed as limiting.

### EXPERIMENTAL SECTION

### Example 1

### Electro Spinning a Solution of Tecogel 2000

Tecogel 2000 was dissolved in a water/ethanol mixture with 20% W/W water and 80% W/W of ethanol. The experiment was carried out with a simple laboratory set-up consisting of a syringe with a stainless steel needle connected to a high-voltage power supply and a grounded aluminum screen placed in the bottom of a paper box as the target. During the spinning process, the current was monitored and the voltage drop across the syringes and in the grounded aluminum screen was kept between 5-10 kV to ground circuit. A small amount of fiber was produced according to this method.

### Example 2

A single screw extruder was used to produce fibres/monofilaments of Tecogel 2000 by melt spinning. The inlet temperature of the extruder was 120° C. The temperature was gradually increased through the extruder and the outlet temperature was 160° C. The fibres were spun into a cooling bath (room temperature) containing a suitable non-solvent like hexane, heptane or aliphatic alcohols. The fibres were afterwards dried and wound using a suitable bobbin speed.

### Example 3

A twin-screw extruder was used to produce fibres of various hydrophilic polyurethanes or mixtures of those by melt spinning. The polymer granulates were feed into the extruder by one or two gravimetric feeders and the temperature was gradually increased through the extruder The polymer melts were stretched to the desired thickness while cooled with air and at last winded on a roll. The thickness of the fibres was controlled by the speed of the winder and the rate of the feeders.

Fibres of the following hydrophilic polyurethane's were produced.

| | | Temperature | | Winder |
|---|---|---|---|---|
| | Ratio | profile in | Feed | speed |
| Polymer(s) | w/w | extruder | rate | 1-10 |
| Hydroslip C | 100 | 130° C.-150° C. | 75 g/h | 10 |
| Tecogel 500 | 100 | 130° C.-150° C. | 75 g/h | 8 |
| Tecogel 2000 | 100 | 130° C.-150° C. | 75 g/h | 8 |
| Tecophilic HP-93A-100 | 100 | 150° C.-190° C. | 80 g/h | 10 |
| Tecogel2000/Tecophilic | 50/50 | 150° C.-170° C. | 130 g/h | 10 |
| HP-93A-100 | | | | |
| Tecogel2000/Tecophilic | 40/60 | 150° C.-180° C. | 160 g/h | 10 |
| HP-93A-100 | | | | |

1: An absorbent fibrous material comprising fibres prepared by melt spinning, solvent spinning, dry spinning or electro spinning of a polymer composition having a water absorption capacity of at least 10 g/g polymer composition and comprising a thermoplastic hydrophilic block copolymer having in itself a water absorption capacity of at least 10 g/g polymer.

2: An absorbent non-woven fibrous material comprising the fibrous material according to claim 1.

3: An absorbent non-woven fibrous material comprising cartable fibres or a woven fibrous material comprising spun fibres wherein said fibres is made from a polymer composition having a water absorption capacity of at least 10 g/g polymer composition and comprising a thermoplastic hydrophilic block copolymer having in itself a water absorption capacity of at least 10 g/g polymer.

4: The absorbent fibrous material according to claim 1, wherein the thermoplastic hydrophilic block copolymer has a water absorption capacity between 10 and 30 g/g polymer.

5: The absorbent fibrous material according to claim 1 comprising fibres prepared by melt spinning.

6: The absorbent fibrous material according to claim 1 comprising fibres prepared by dry spinning.

7: The absorbent fibrous material according to claim 1 comprising fibres prepared by solvent spinning.

8: The absorbent fibrous material according to claim 1 comprising fibres prepared by electro spinning.

9: The absorbent fibrous material according to claim 3 wherein the fibres has been prepared by melt spinning, solvent spinning or dry spinning of the polymer composition.

10: The absorbent fibrous material according to claim 1 wherein the polymer composition comprises the thermoplastic hydrophilic block copolymer and one or more hydrophilic homopolymers or heteropolymers.

11: The absorbent fibrous material according to claim 1 wherein the polymer composition comprises one or more thermoplastic hydrophilic block copolymer(s).

12: The absorbent fibrous material according to claim 1 wherein the thermoplastic hydrophilic block copolymer is an amphiphilic block copolymer containing hydrophobic as well as hydrophilic block(s).

13: The absorbent fibrous material according to claim 1 wherein the absorbent fibrous material comprises the fibres made from said polymer composition as well as other fibres.

14: The absorbent fibrous material according to claim 13 wherein the absorbent fibrous material comprises the fibres made from said polymer composition as well as gel-forming fibres, super-absorbent fibres, and/or fibres providing strength or elasticity to the fibrous material.

15: The absorbent fibrous material according to claim 13 comprising at least 20% w/w of fibres made of the polymer composition, more preferred at least 30% w/w and most preferred at least 50% w/w of fibres made of the polymer composition.

16: The absorbent fibrous material according to claim 1 wherein the fibrous material has a water absorption capacity of at least 10 g/g fibrous material.

17: The absorbent fibrous material according to claim 1 wherein the hydrophilic block copolymer is a hydrophilic or amphiphilic polyurethane, such as Tecogel TG 2000, HydroMed D640™ or HydroSlip C™.

18: The fibrous material according to claim 12 wherein the hydrophobic block(s) comprises monovinyl aromatic monomers, containing from about 8 to about 18 carbon atoms, such as styrene, alpha-methylstyrene, vinyltoluene, vinylpyridine, ethylstyrene, t-butylstyrene, isopropylstyrene, dimethylstyrene, and other alkylated styrenes.

19: The fibrous material according to claim 18 wherein the hydrophilic block(s) comprises ethylenically unsaturated carboxylic acids, such as acrylic acid, methacrylic acid, itaconic acid, maleic acid or fumaric acid.

20: The fibrous material according to claim 19 wherein the hydrophobic block is polystyrene and the hydrophilic block is polyacrylic acid.

21: The fibrous material according to claim 18 wherein the hydrophilic block(s) comprises ethylene oxide.

22: The fibrous material according to claim 21 wherein the hydrophobic block is polystyrene and the hydrophilic block is polyethylene glycol.

23: The fibrous material according to claim 12 wherein the hydrophobic block comprises ethylenically unsaturated monomers, such as butadiene, chloroprene, (meth)acrylic esters, vinyl esters; or vinyl halides or vinyl nitrites.

24: The fibrous material according to claim 23 wherein the hydrophilic block(s) comprises ethylenically unsaturated monocarboxylic acids, such as acrylic acid, methacrylic acid, itaconic acid, maleic acid and fumaric acid.

25: The fibrous material according to claim 24 wherein the hydrophobic block is a poly(meth)acrylic ester, such as polymethylmethacrylate, and the hydrophilic block(s) is polymethacrylic acid.

26: The fibrous material according to claim 12 wherein the block copolymer is a tri-block copolymer of the type A-B-A, wherein A is hydrophobic block(s) and B is a hydrophilic block

27: A wound dressing comprising an absorbent fibrous material according to claim 1.

28: The wound dressing according to claim 27 wherein the wound dressing consist of said absorbent fibrous material.

29: The wound dressing according to claim 27 which contain said absorbent fibrous material in addition to other elements.
